(19) United States
(12) Patent Application Publication
(10) Pub. No.: US 2024/0374930 A1
(43) Pub. Date: Nov. 14, 2024

(54) TREATMENT SUPPORT SYSTEM, TREATMENT SUPPORT METHOD, AND TREATMENT SUPPORT PROGRAM

(71) Applicants: OSAKA UNIVERSITY, Osaka (JP); MIZUHO RESEARCH & TECHNOLOGIES, LTD., Tokyo (JP)

(72) Inventors: Teiji NISHIO, Suita-shi (JP); Yuya Nemoto, Tokyo (JP); Hidemasa MAEKAWA, Tokyo (JP)

(21) Appl. No.: 18/578,283

(22) PCT Filed: Jun. 24, 2022
(86) PCT No.: PCT/JP2022/025351

§ 371 Date: Jan. 10, 2024

(30) Foreign Application Priority Data
Jul. 16, 2021 (JP) 2021-118224

(51) Int. Cl. A61N 5/10

(52) U.S. Cl. CPC A61N 5/1065; A61N 5/1071; A61N 2005/1087

(57) ABSTRACT

A support apparatus includes an irradiation device for emitting a particle beam, a detection device for detecting a particle beam irradiation region, and a control unit for acquiring the irradiation region. The control unit specifies an attention region within the irradiation region in a treatment plan for a patient, and specifies the irradiation position for pre-irradiation within the attention region. The control unit instructs the irradiation device to carry out pre-irradiation with irradiation energy in the treatment plan at the irradiation position, and acquires the irradiation region detected by the detection device. The control unit further adjusts irradiation conditions in the treatment plan according to the irradiation region in the pre-irradiation and instructs the irradiation device to carry out post-irradiation under the adjusted irradiation conditions.

### TECHNICAL FIELD

The present disclosure relates to a treatment support system, a treatment support method, and a treatment support program for supporting treatment using a particle beam.

### BACKGROUND ART

When treatment is performed using radiation, it is necessary to irradiate an appropriate position with an appropriate amount of radiation in order to reduce a radiation exposure level of a patient. For this reason, a radiation treatment control device that calculates a current position of a marker embedded in a patient and reduces a radiation exposure level of the patient due to an imaging radiation emitted from a fluoroscopic imaging device for fluoroscopy during radiation treatment has been studied (see, for example, Patent Literature 1). In a technique described in the literature, fluoroscopic images of three or more markers are acquired from a set of fluoroscopic imaging devices, and distances between the markers are acquired. Then, a current position of each marker is calculated, and it is determined whether or not to emit treatment radiation.

In addition, treatment may be performed using a high-speed particle beam with high energy. A proton beam, which is one of particle beams, loses a large amount of energy at a location immediately before the incident proton stops in a body, and forms a high dose area called “Bragg peak” at the location. Therefore, it is possible to reduce the damage of a normal area and intensively irradiate an affected part in a body with strong radiation. In such irradiation with a particle beam (proton beam), a technique of visualizing an area irradiated with a proton beam (hereinafter called irradiation area) by using a nuclear spallation reaction occurring by an incident proton nucleus and a target nucleus in a patient body, and deriving an irradiation dose to a tumor from the visualized information has also been studied (see, for example, Non-Patent Literature 1). In the technique described in this literature, a positron emission tomography device (PET device) “Beam ON-LINE Positron Emission Tomography system” that detects a positron emission nucleus generated in an irradiation area in a patient body by a target nuclear spallation reaction in a proton beam treatment is used. This PET device is installed in a proton beam rotary gantry irradiation chamber to visualize a proton beam irradiation area.

### CITATIONS LIST

### Patent Literature

Patent Literature 1: JP 2013-192702 A

### Non-Patent Literature

Non-Patent Literature 1: Sadaharu Nishio, “Innovative proton therapy with irradiated volume image by target nuclear fragment reaction”, [online], 2011, Research Organization for Information Science and Technology, RIST News, No. 50, p. 24-35, [searched on Jun. 14, 2021], Internet <URL: http://www.rist.or.jp/rnews/50/50s4.pdf>

### SUMMARY OF INVENTION

### Technical Problem

However, since dose distribution changes according to a state around an affected part of a patient, accurate irradiation may be difficult. For example, when an important organ exists around the affected part, it is important to reduce an influence of a deviation of an irradiation position of the particle beam.

### Solution to Problem

In one aspect, a treatment support system is provided. The treatment support system includes an irradiation device configured to emit a particle beam, a detection device configured to detect an irradiation area of the particle beam, and a control unit configured to acquire the irradiation area. The control unit is configured to identify an attention area within an irradiation range in a treatment plan for a patient, specify an irradiation position for first irradiation in the attention area, instruct the irradiation device to perform the first irradiation with an irradiation energy in the treatment plan at the irradiation position, acquire an irradiation area of the first irradiation detected by the detection device, adjust an irradiation condition of the treatment plan depending on the irradiation area of the first irradiation, and instruct the irradiation device to perform second irradiation under the adjusted irradiation condition.

In another aspect, a method for supporting treatment by using a treatment support system is provided. The system includes an irradiation device configured to emit a particle beam, a detection device configured to detect an irradiation area of the particle beam, and a control unit configured to acquire the irradiation area. The method includes identifying, by the control unit, an attention area within an irradiation range in a treatment plan for a patient, specifying, by the control unit, an irradiation position for first irradiation in the attention area, instructing, by the control unit, the irradiation device to perform the first irradiation with an irradiation energy in the treatment plan at the irradiation position, acquiring, by the control unit, an irradiation area of the first irradiation detected by the detection device, adjusting, by the control unit, an irradiation condition of the treatment plan depending on the irradiation area of the first irradiation, and instructing, by the control unit, the irradiation device to perform second irradiation under the adjusted irradiation condition.

In yet another aspect, a program for supporting treatment by using a treatment support system is provided. The system includes an irradiation device configured to emit a particle beam, a detection device configured to detect an irradiation area of the particle beam, and a control unit configured to acquire the irradiation area. The program, when executed by the control unit, causes the control unit to identify an attention area within an irradiation range in a treatment plan for a patient, specify an irradiation position for first irradiation in the attention area, instruct the irradiation device to perform the first irradiation with an irradiation energy in the treatment plan at the irradiation position, acquire an irradiation area of the first irradiation detected by the detection device, adjust an irradiation condition of the treatment plan depending on the irradiation area of the first irradiation, and instruct the irradiation device to perform second irradiation under the adjusted irradiation condition.

### Advantageous Effects of Invention

According to the present disclosure, it is possible to support treatment by irradiation with a particle beam in consideration of a surrounding situation of an affected part.

### BRIEF DESCRIPTION OF DRAWINGS

FIG. 1 is an explanatory diagram of a treatment support system according to an embodiment.

FIG. 2 is an explanatory diagram of a hardware configuration according to the embodiment.

FIG. 3 is an explanatory diagram of a processing procedure according to the embodiment.

FIG. 4 is an explanatory diagram of an irradiation position of a particle beam according to the embodiment.

FIG. 5 is an explanatory diagram of a pre-irradiation position according to the embodiment.

FIG. 6 is an explanatory diagram of an irradiation area according to the embodiment.

### DESCRIPTION OF EMBODIMENTS

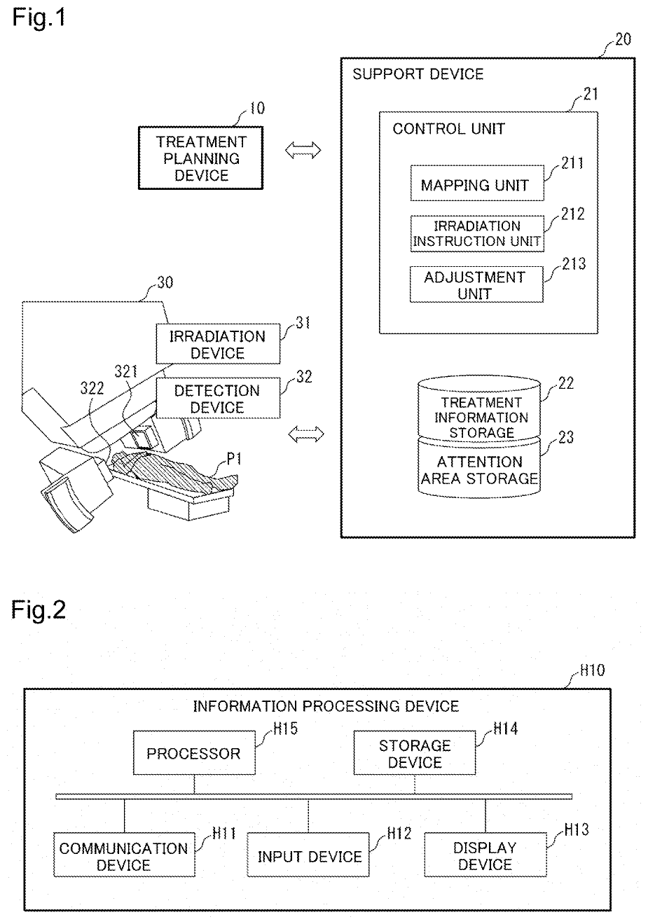

Hereinafter, an embodiment embodying a treatment support system, a treatment support method, and a treatment support program will be described with reference to FIGS. 1 to 6. In the present embodiment, a case where an affected part of a patient is irradiated with protons as particle beams to treat the affected part will be described. In this case, pre-irradiation (first irradiation) for confirming an irradiation state of the proton beam and post-irradiation (second irradiation) for treating the affected part are performed.

Therefore, a treatment planning device 10, a support device 20, and a treatment device 30 connected via a network are used.

### Hardware Configuration Example

FIG. 2 is a hardware configuration example of an information processing device H10 that functions as the treatment planning device 10, the support device 20, the treatment device 30, and the like.

The information processing device H10 includes a communication device H11, an input device H12, a display device H13, a storage device H14, and a processor H15. This hardware configuration is an example, and may include another hardware.

The communication device H11 is an interface that establishes a communication path with another device to transmit and receive data, and is, for example, a network interface, a wireless interface, or the like.

The input device H12 is a device that accepts input from a user or the like, and is, for example, a mouse, a keyboard, or the like. The display device H13 is a display, a touch panel, or the like that displays various types of information.

The storage device H14 is a storage device that stores data and various programs for executing various functions of the treatment planning device 10, the support device 20, and the treatment device 30. Examples of the storage device H14 include a non-transitory computer-readable medium such as a ROM, a RAM, and a hard disk.

The processor H15 controls each processing (for example, processing in a control unit 21 to be described later) in the treatment planning device 10, the support device 20, and the treatment device 30 by using programs and data stored in the storage device H14. Examples of the processor H15 include a CPU, an MPU, and the like. The processor H15 deploys, in the RAM, the programs stored in the ROM or the like, and operates to execute various types of processing. For example, in a case where application programs of the treatment planning device 10, the support device 20, and the treatment device 30 are activated, the processor H15 operates to execute each processing described later.

The processor H15 is not limited to a processor that performs software processing for all processing executed by the processor. For example, the processor H15 may include a dedicated hardware circuit (e.g., application specific integrated circuit: ASIC) that performs hardware processing for at least some of processing executed by the processor H15. That is, the processor H15 can be configured by the following constituents.
[1] One or more processors operating according to computer programs,
[2] One or more dedicated hardware circuits executing at least some of various types of processing, or
[3] Circuitry including a combination thereof.

The processor includes a CPU and a memory such as a RAM and a ROM, and the memory stores a program code or a command configured to cause the CPU to execute processing. The memory, i.e., a computer-readable medium, includes any available medium that can be accessed by a general-purpose or dedicated computer.

### Functions of Each Information Processing Device

Next, functions of the treatment planning device 10, the support device 20, and the treatment device 30 will be described.

The treatment planning device 10 is a simulator for examining a method of incident radiation on an affected part and confirming whether an appropriate dose has been prescribed. The treatment planning device 10 acquires, from a CT imaging device, CT images (DICOM data) for which tomography is performed at predetermined image intervals. Then, the treatment planning device 10 performs contour extraction in DICOM data using a known method, to generate CT contour information. The CT contour information includes DICOM Region of Interest (ROI) data, and is data including an aggregate of points (coordinates) forming a contour of a predetermined part (body surface, bone, affected part, organ at risk, or the like) specified in a CT image (tomographic image) captured at predetermined intervals. In the treatment planning device 10, a quality of treatment beam, an incident direction, an irradiation range, a prescribed dose, the number of times of irradiation, and the like are determined depending on a body surface shape of the affected part, a shape and position of the affected part, and a positional relationship with an organ at risk.

The support device 20 is a computer system for supporting particle beam (proton beam) treatment. The support device 20 includes a control unit 21, a treatment information storage 22, and an attention area storage 23.

The control unit 21 performs processing (processing including mapping stage, irradiation instruction stage, adjustment stage, or the like) to be described later. By executing the treatment support program for this purpose, the control unit 21 functions as a mapping unit 211, an irradiation instruction unit 212, an adjustment unit 213, and the like.

The mapping unit 211 executes processing of determining a position at which pre-irradiation of the proton beam is performed.

The irradiation instruction unit 212 executes processing of instructing the treatment device 30 to emit a proton beam.

The adjustment unit 213 executes processing of adjusting an irradiation condition of the post-irradiation of the proton beam according to a result of the pre-irradiation.

The treatment information storage 22 stores treatment management information regarding proton beam irradiation for treatment of a patient. When the support device 20 acquires treatment plan information from the treatment planning device 10, the treatment information storage 22 stores acquired treatment management information. The treatment management information includes CT contour information and irradiation condition information in association with a patient code and a scheduled treatment date.

The patient code is an identifier for identifying each patient.

The scheduled treatment date is a scheduled date (year/month/day) of treatment by proton beam irradiation for the patient in a treatment plan.

The CT contour information includes position information on a contour of a predetermined part (body surface, bone, affected part, organ at risk, or the like) in a CT image of an affected part of the patient.

The irradiation condition is a condition for irradiating the patient with a proton beam on the scheduled treatment date. Examples of the irradiation condition include a proton beam irradiation position, an irradiation direction, irradiation energy, an irradiation dose, a beam irradiation method, and the like. Examples of the beam irradiation method include an “extended beam irradiation method” and a “scanning irradiation method”.

The attention area storage 23 stores attention area management information for specifying an attention area. When a detection condition for specifying an attention area is determined, the attention area storage 23 stores the attention area management information. The attention area management information includes information related to a detection condition and a score for specifying the attention area.

In the present embodiment, for example, the following areas are specified as attention areas.

(a) Area having large change in composition or density

For example, an area of a CT image where distribution unevenness (complexity) of the CT value is large due to a mixture of tissue of a human body (bone or the like).

(b) Area with possible composition change

An area where a composition change may occur depending on a condition of a patient (for example, presence or absence of nasal discharge).

(c) Area where a patient is greatly affected when irradiation position is shifted

An area where an organ at risk (important organ such as optic nerve tissue and brain tissue) exists in the vicinity of the irradiation range.

(d) Area with small irradiation dose

An area where an irradiation dose of proton beam is small in an irradiation condition of the treatment plan. In the area where an irradiation dose is small, an influence of the pre-irradiation dose is large, and it is difficult to adjust the post-irradiation dose.

The score is a value given to an attention area detected under each detection condition. Here, for attention areas a to c, a higher score is set so as to promote the confirmation by the pre-irradiation for the attention area in which attention should be paid to the post-irradiation. In addition, a score for suppressing pre-irradiation is set for the attention area d.

The treatment device 30 is a device that irradiates an affected part with radiation to treat the affected part such as cancer. The treatment device 30 is provided with a treatment table on which a patient P1 lies on his/her back or supinely. The treatment device 30 includes an irradiation device 31 and a detection device 32.

The irradiation device 31 is a device (rotary gantry) that irradiates the patient P1 on the treatment table with radiation from 360-degree arbitrary direction.

The detection device 32 is a positron emission tomography device (PET device) that detects a positron emission nucleus generated in an irradiation area in a patient body by a target nuclear spallation reaction in proton beam treatment. An irradiation depth position (irradiation area) can be specified by an emission position of the positron emission nucleus. The detection device 32 includes measurement surfaces 321, 322 that detect a positron emission nucleus on a side surface in an irradiation direction of the proton beam emitted from the irradiation device 31.

### Irradiation Support Processing

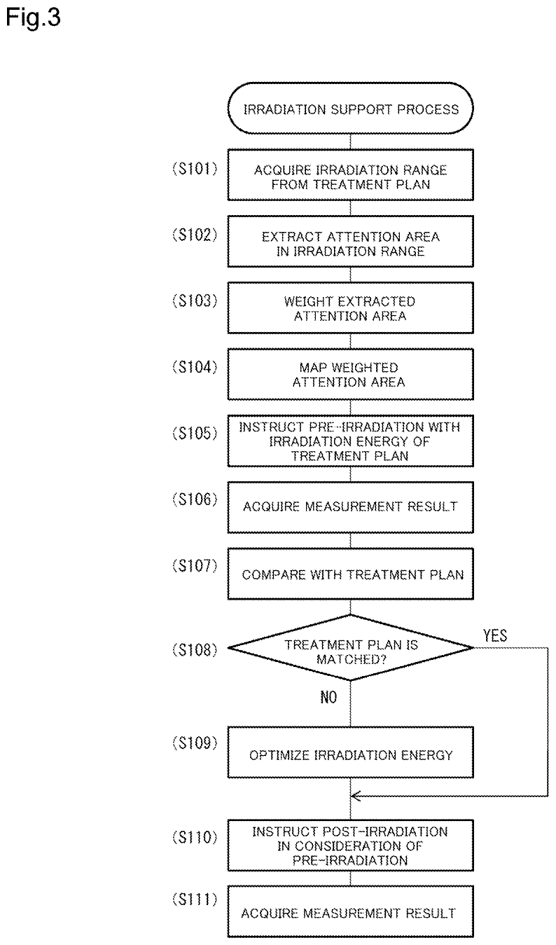

The irradiation support processing will be described with reference to FIG. 3.

First, the control unit 21 of the support device 20 executes processing of acquisition of an irradiation range from the treatment plan (step S101). Specifically, the mapping unit 211 of the control unit 21 acquires treatment plan information corresponding to a patient code of a patient to be treated by proton beam irradiation on a scheduled treatment date (current day) from the treatment planning device 10, and stores the treatment plan information in the treatment information storage 22. Then, the mapping unit 211 specifies an irradiation range of the proton beam using an irradiation condition (irradiation position, irradiation direction, and irradiation energy of proton beam) in the CT contour information.

Next, the control unit 21 of the support device 20 executes extraction processing of an attention area in the irradiation range (step S102). Specifically, the mapping unit 211 of the control unit 21 maps an attention area greatly affected by a deviation of the irradiation position in the CT contour information on the irradiation range of the particle beam using the attention area storage 23.

For the detection condition “area having large change in composition or density”, an index indicating distribution unevenness of the CT value is calculated for each area included in the image by image analysis of the CT image. Then, an area where the index is equal to or more than a distribution unevenness reference value is specified.

In addition, for the detection condition “area with possible composition change”, an area where a composition may change (for example, nasal cavity) is specified according to a state of a patient by image analysis of the CT image.

Furthermore, for the detection condition “area where a patient is greatly affected when irradiation position is shifted”, an area where an organ at risk is present (for example, optic nerve tissue or brain tissue) is specified by image analysis of the CT image.

For the detection condition “area with small irradiation dose”, an area where an irradiation dose of the proton beam is smaller than a dose reference value under an irradiation condition of a treatment plan is specified.

Next, the control unit 21 of the support device 20 executes weighting processing of an extracted attention area (step S103). Specifically, the mapping unit 211 of the control unit 21 performs weighting by scoring obtained by adding a score of the attention area storage 23 for each attention area mapped under a detection condition.

Next, the control unit 21 of the support device 20 executes a mapping process of the weighted attention area (step S104). Specifically, the mapping unit 211 of the control unit 21 specifies (maps) an attention area having a high score within an irradiation range and its vicinity range (predetermined distance range) of a treatment plan. Next, the mapping unit 211 determines a pre-irradiation position of the proton beam by pencil-beam approximation in an attention area weighted by a high score. For example, a case is assumed in which a head is irradiated with a plurality of proton beams.

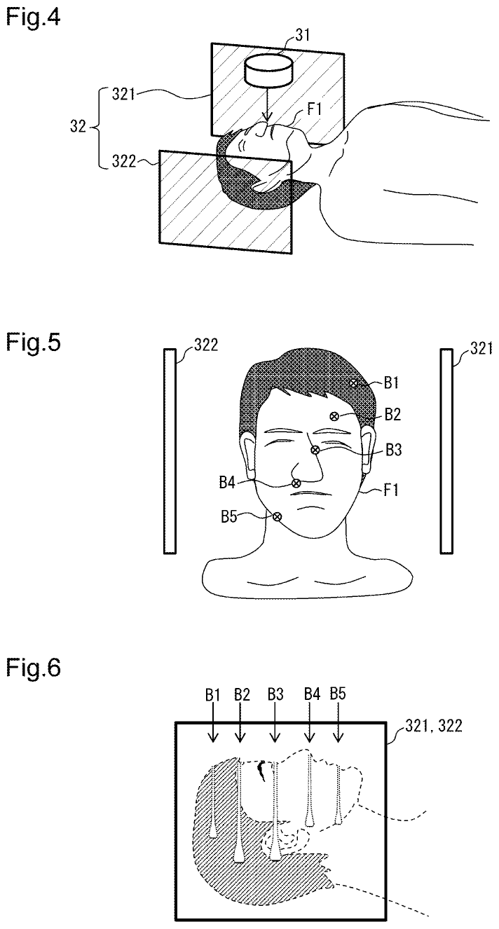

As illustrated in FIG. 4, a case is assumed in which the proton beams are emitted from the irradiation device 31 toward a front of a face F1. In this case, the measurement surface 321, 322 of the detection device 32 are arranged such that the proton beams can be detected along an irradiation direction.

Then, as illustrated in FIG. 5, pre-irradiation positions B1, B2, B3, B4, and B5 are determined at positions that do not overlap when viewed from normal directions of the measurement surfaces 321, 322.

Next, the control unit 21 of the support device 20 executes processing of pre-irradiation instruction with an irradiation energy of the treatment plan (step S105). Specifically, the irradiation instruction unit 212 of the control unit 21 transmits a pre-irradiation instruction to the treatment device 30 that performs proton beam irradiation for each pre-irradiation position. The pre-irradiation instruction includes information on an irradiation energy of the proton beam and a pre-irradiation dose (scheduled value). In this case, as for the irradiation energy, the irradiation energy at each pre-irradiation position in the treatment plan is set. In addition, as for the pre-irradiation dose (scheduled value), the minimum dose that is within the prescribed dose of the treatment plan and can detect a positron emission nucleus by the detection device 32 is used. Then, the irradiation device 31 of the treatment device 30 irradiates each instructed pre-irradiation position with the proton beam using the irradiation energy and the pre-irradiation dose.

Next, the control unit 21 of the support device 20 executes acquisition processing of a measurement result (step S106). Specifically, the adjustment unit 213 of the control unit 21 detects a positron emission nucleus by the pre-irradiation from the detection device 32 of the treatment device 30, and acquires the irradiation depth position information (irradiation area) and the pre-irradiation dose (actual value) from the emission distribution.

In this case, as illustrated in FIG. 6, a position of a high dose area (actual irradiation depth) where the particle beam of each of the pre-irradiation positions B1 to B5 has reached is detected by the measurement surfaces 321, 322.

Next, the control unit 21 of the support device 20 executes comparison processing between the measurement result and the treatment plan (step S107). Specifically, the adjustment unit 213 of the control unit 21 compares a scheduled depth position and a pre-irradiation dose (scheduled value) in pencil beam approximation using the irradiation energy with an actual depth position and a pre-irradiation dose (actual value) detected by the detection device 32.

Next, the control unit 21 of the support device 20 executes determination processing whether the measurement result matches the treatment plan (step S108). Specifically, the adjustment unit 213 of the control unit 21 determines that the measurement result matches the treatment plan when the scheduled value of the irradiation dose matches the actual value and the scheduled depth position matches the actual depth position. In a case where there is a deviation (difference) between the scheduled depth position and the actual depth position, it is determined that the measurement result does not match the treatment plan.

When it is determined that the measurement result does not match the treatment plan (in a case of “NO” in step S108), the control unit 21 of the support device 20 executes optimization processing of irradiation energy (step S109). Specifically, the adjustment unit 213 of the control unit 21 adjusts the irradiation energy according to the actual depth position. For example, in an area where the actual depth position is deeper than the scheduled depth position, the irradiation energy is lowered depending on the difference. On the other hand, in an area where the actual depth position is shallower than the scheduled depth position, the irradiation energy is increased depending on the difference.

On the other hand, when it is determined that the measurement result matches the treatment plan (in a case of “YES” in step S108), the control unit 21 of the support device 20 skips optimization processing of irradiation energy (step S109).

Next, the control unit 21 of the support device 20 executes processing of post-irradiation instruction in consideration of pre-irradiation (step S110). Specifically, the adjustment unit 213 of the control unit 21 calculates a pre-irradiation dose (irradiated dose) in each irradiation area included in the irradiation range of the treatment plan using an actual depth position and a pre-irradiation dose (actual value). Next, the adjustment unit 213 calculates a post-irradiation dose obtained by subtracting the pre-irradiation dose (irradiated dose) from the irradiation dose of the treatment plan in each irradiation area. Then, the irradiation instruction unit 212 transmits a post-irradiation instruction to the treatment device 30 for each irradiation position. The post-irradiation instruction includes information on an irradiation energy of the proton beam and the post-irradiation dose. Then, the irradiation device 31 of the treatment device 30 irradiates each instructed irradiation position with the proton beam using the irradiation energy and the post-irradiation dose.

Next, the control unit 21 of the support device 20 executes acquisition processing of a measurement result as in step S106 (step S111). Specifically, the adjustment unit 213 of the control unit 21 acquires irradiation area information on post-irradiation from the detection device 32 of the treatment device 30.

According to the present embodiment, the following advantages can be obtained.
(1) In the present embodiment, the control unit 21 of the support device 20 executes extraction processing of an attention area in an irradiation range (step S102). As a result, it is possible to specify an attention area at the time of proton beam irradiation in the treatment plan.
(2) In the present embodiment, the control unit 21 of the support device 20 executes weighting processing of an extracted attention area (step S103). As a result, a priority order of the attention area to be pre-irradiated can be determined by weighting among a plurality of attention areas.
(3) In the present embodiment, the control unit 21 of the support device 20 executes mapping processing of the weighted attention area (step S104). Pre-irradiation positions B1 to B5 are determined at positions not overlapping with each other as viewed from the normal directions of the measurement surfaces 321, 322. As a result, it is possible to specify the irradiation area at each pre-irradiation position from the side surface in an irradiation direction while reducing a pre-irradiation dose in a state where irradiation areas of a positron emission nucleus do not overlap each other.
(4) In the present embodiment, the control unit 21 of the support device 20 executes pre-irradiation instruction processing (step S105) and measurement result acquisition processing (step S106) with the irradiation energy of the treatment plan. As a result, it is possible to recognize an irradiation situation of the proton beam with a dose smaller than the prescribed dose. Then, irradiation conditions in the post-irradiation can be finely adjusted. In particular, when a large dose of irradiation is performed in a short time, it is necessary to perform irradiation at an accurate position with an appropriate dose, and it is effective to adjust irradiation conditions of post-irradiation by pre-irradiation.
(5) In the present embodiment, when it is determined that the measurement result does not match the treatment plan (in a case of “NO” in step S108), the control unit 21 of the support device 20 executes optimization processing of irradiation energy (step S109). As a result, irradiation according to the treatment plan can be performed in consideration of a patient's situation and the like.
(6) In the present embodiment, the control unit 21 of the support device 20 executes processing of post-irradiation instruction in consideration of pre-irradiation (step S110). As a result, proton beam irradiation of the prescribed dose of the treatment plan can be performed in consideration of the pre-irradiation.
(7) In the present embodiment, the control unit 21 of the support device 20 executes acquisition processing of a measurement result (step S111). As a result, it is possible to confirm whether there is a deviation between a scheduled irradiation area and an actual irradiation area in the treatment plan.

The present embodiment can be modified to be implemented as follows. The present embodiment and the following modification examples can be implemented in combination with each other within a range not technically inconsistent.

In the above embodiment, a proton beam is used as a particle beam. The particle beam is not limited to the proton beam, and for example, a carbon beam or the like can also be used.

In the above embodiment, the control unit 21 of the support device 20 executes mapping processing of the weighted attention area (step S104). Five pre-irradiation positions are determined at positions not overlapping with each other as viewed from the normal directions of the measurement surfaces. The number of pre-irradiation positions is not limited to five.

In the above embodiment, the attention area storage 23 stores detection conditions (a) to (d) for specifying an attention area. The detection conditions are not limited to these conditions, and some of these conditions or other conditions may be used.

In addition, a template in which the attention area is mapped for each body part may be prepared, and the attention area may be specified by fitting to a contour of a patient.

In the above embodiment, the detection device 32 is a positron emission tomography device, and detects a positron emission nucleus generated in an irradiation area in a patient body. As long as the irradiation area can be detected, the detection is not limited to the detection of the positron emission nucleus.

1. A treatment support system comprising:
an irradiation device configured to emit a particle beam;
a detection device configured to detect an irradiation area of the particle beam; and
circuitry, wherein
the circuitry is configured to
identify an attention area within an irradiation range in a treatment plan for a patient,
specify an irradiation position for first irradiation in the attention area,
instruct the irradiation device to perform the first irradiation with an irradiation energy in the treatment plan at the irradiation position,
acquire an irradiation area of the first irradiation detected by the detection device,
adjust an irradiation condition of the treatment plan depending on the irradiation area of the first irradiation, and
instruct the irradiation device to perform second irradiation under the adjusted irradiation condition.

2. The treatment support system according to claim 1, wherein the circuitry is configured to specify the attention area in the irradiation range on a basis of a detection condition depending on complexity of tissue of a human body.

3. The treatment support system according to claim 1, wherein the circuitry is configured to specify the attention area in the irradiation range on a basis of a detection condition corresponding to a possibility of a composition change caused by a state of the patient.

4. The treatment support system according to claim 1, wherein the circuitry is configured to specify the attention area in the irradiation range on a basis of a detection condition corresponding to presence of an organ.

5. The treatment support system according to claim 1, wherein the attention area is a first attention area among a plurality of attention areas, and
the circuitry is configured to score the plurality of attention areas depending on a detection condition, and specify an attention area where the first irradiation is performed depending on a result of the scoring.

6. The treatment support system according to claim 1, wherein the detection device has a measurement surface that measures the irradiation area,
the irradiation position is a first irradiation position among a plurality of irradiation positions, and
the circuitry is configured to determine the plurality of irradiation positions such that an irradiation direction of the particle beam of the first irradiation at each irradiation position does not overlap with other irradiation positions of the plurality of irradiation positions when viewed from a normal direction of the measurement surface.

7. A treatment support method for supporting treatment by using a treatment support system, the system comprising:
an irradiation device configured to emit a particle beam;
a detection device configured to detect an irradiation area of the particle beam; and
a circuitry configured to acquire the irradiation area, the method comprising:
identifying, by the circuitry, an attention area within an irradiation range in a treatment plan for a patient;
specifying, by the circuitry, an irradiation position for first irradiation in the attention area;
instructing, by the circuitry, the irradiation device to perform the first irradiation with an irradiation energy in the treatment plan at the irradiation position;
acquiring, by the circuitry, an irradiation area of the first irradiation detected by the detection device;
adjusting, by the circuitry, an irradiation condition of the treatment plan depending on the irradiation area of the first irradiation; and
instructing, by the circuitry, the irradiation device to perform second irradiation under the adjusted irradiation condition.

8. A non-transitory computer-readable medium that stores a treatment support program for supporting treatment by using a treatment support system, the system comprising:
an irradiation device configured to emit a particle beam;
a detection device configured to detect an irradiation area of the particle beam; and
a circuitry configured to acquire the irradiation area, wherein the treatment support program, when executed by the circuitry, causes the circuitry to
identify an attention area within an irradiation range in a treatment plan for a patient;
specify an irradiation position for first irradiation in the attention area;
instruct the irradiation device to perform the first irradiation with an irradiation energy in the treatment plan at the irradiation position;
acquire an irradiation area of the first irradiation detected by the detection device;
adjust an irradiation condition of the treatment plan depending on the irradiation area of the first irradiation; and
instruct the irradiation device to perform second irradiation under the adjusted irradiation condition.

9. The treatment support method according to claim 7, further comprising specifying the attention area in the irradiation range on a basis of a detection condition depending on complexity of tissue of a human body.

10. The treatment support method according to claim 7, further comprising specifying the attention area in the irradiation range on a basis of a detection condition corresponding to a possibility of a composition change caused by a state of the patient.

11. The treatment support method according to claim 7, further comprising specifying the attention area in the irradiation range on a basis of a detection condition corresponding to presence of an organ.

12. The treatment support method according to claim 7, wherein the attention area is a first attention area among a plurality of attention areas, and
the method further comprises
scoring the plurality of attention areas depending on a detection condition, and
specifying an attention area where the first irradiation is performed depending on a result of the scoring.

13. The treatment support method according to claim 7, wherein the detection device has a measurement surface that measures the irradiation area,
the irradiation position is a first irradiation position among a plurality of irradiation positions, and
the method further comprises
determining the plurality of irradiation positions such that an irradiation direction of the particle beam of the first irradiation at each irradiation position does not overlap with other irradiation positions of the plurality of irradiation positions when viewed from a normal direction of the measurement surface.

14. The non-transitory computer-readable medium according to claim 8, wherein the program further causes the circuitry to specify the attention area in the irradiation range on a basis of a detection condition depending on complexity of tissue of a human body.

15. The non-transitory computer-readable medium according to claim 8, wherein the program further causes the circuitry to specify the attention area in the irradiation range on a basis of a detection condition corresponding to a possibility of a composition change caused by a state of the patient.

16. The non-transitory computer-readable medium according to claim 8, wherein the program further causes the circuitry to specify the attention area in the irradiation range on a basis of a detection condition corresponding to presence of an organ.

17. The non-transitory computer-readable medium according to claim 8, wherein the attention area is a first attention area among a plurality of attention areas, and
the program further causes the circuitry to
score the plurality of attention areas depending on a detection condition, and
specify an attention area where the first irradiation is performed depending on a result of the scoring.

18. The non-transitory computer-readable medium according to claim 8, wherein the detection device has a measurement surface that measures the irradiation area,
the irradiation position is a first irradiation position among a plurality of irradiation positions, and
the program further causes the circuitry to determine the plurality of irradiation positions such that an irradiation direction of the particle beam of the first irradiation at each irradiation position does not overlap with other irradiation positions of the plurality of irradiation positions when viewed from a normal direction of the measurement surface.
